(19) United States
(12) Patent Application Publication
(10) Pub. No.: US 2021/0077082 A1
(43) Pub. Date: Mar. 18, 2021

(54) Biopsy Sampling Method of a Target Tissue of a Human Body Through a Natural Passage of the Human Body

(71) Applicant: Shengsuo Biotech (Shanghai) Corp., Ltd., Shanghai (CN)

(72) Inventors: Changgeng Liu, Shanghai (CN); Bo Xiao, Shanghai (CN)

(21) Appl. No.: 17/004,386

(22) Filed: Aug. 27, 2020

(30) Foreign Application Priority Data
Sep. 18, 2019 (CN) 201910883332.1
Dec. 13, 2019 (CN) 201911285767.2

(51) Int. Cl. A61B 10/04; A61B 8/12

(52) U.S. Cl. CPC A61B 10/04; A61B 2017/3413; A61B 8/12

(57) ABSTRACT

A biopsy and ultrasound combined device comprises a handle body, a depth adjustment portion, an outer sleeve, a biopsy needle bar, and an ultrasonic probe. The biopsy and ultrasound combined device accommodates the ultrasonic probe in the biopsy needle bar, which is accommodated in the outer sleeve. The depth adjustment portion is movably connected to the handle body and fixedly connected to the ultrasonic probe and the biopsy needle bar, respectively, so that the ultrasonic probe, the outer sleeve and the biopsy needle bar are free to move, thereby facilitating accurate positioning for a puncture direction by the ultrasonic probe before a puncture, and further facilitating confirming that the puncture penetrates a target tissue after the puncture. The biopsy needle bar and at least a portion of the insulating intervention sleeve have flexibility and a hardness of the insulating connecting sleeve is greater than that of the insulating puncture sleeve.

### FIELD OF TECHNOLOGY

The invention relates to a technical field of medical instruments, and more specifically, to a biopsy and ultrasound combined device.

### BACKGROUND

Puncture biopsy is a routine method for tumor treatment. After the biopsy needle is inserted into the diseased region, part of the diseased tissue is taken for subsequent pathological analysis. The final treatment scheme depends largely on the results of pathological analysis. Therefore, the prior art generally uses a puncture biopsy process in conjunction with a medical imaging device to jointly locate a diseased region, so as to improve the accuracy of the results of the pathological analysis.

Among the commonly used medical imaging devices, since the fiber-type ultrasonic probe applied to the ultrasound endoscope has an ultrasound transducer fixed at the operation end, it can penetrate deep into the internal part of the human body through artificial channels or natural channels of the human body, such as a urinary cavity. Driven by an external motor, the ultrasound transducer is free to rotate within the ultrasonic probe to create a circular or cross-sectional image of the tissue section perpendicular to the axial direction, thereby obtaining a clear image of the lesion.

Chinese patent application publication No. CN102056559A discloses a biopsy device with an acoustic element. The biopsy device has a needle bar, and different parts of the needle bar are provided with relatively fixed transducer elements. That is, the image information obtained by the transducer return signal depends on the moving position of the needle bar and the arrangement of the transducer elements, which greatly limits the range of image acquisition of transducer elements, and easily leads to missed diagnosis and mis-diagnosis.

Therefore, it is necessary to develop a new type of biopsy and ultrasound combined device to avoid the above problems in the prior art.

### SUMMARY

The purpose of the invention is to provide a biopsy and ultrasound combined device to improve the success rate of biopsy sampling and the accuracy of biopsy results.

The biopsy and ultrasound combined device of the invention comprises a handle body, a depth adjustment portion, an outer sleeve, a biopsy needle bar, and an ultrasonic probe. The outer sleeve has an insulating intervention sleeve and an insulating connecting sleeve that are fixedly connected to each other. The biopsy needle bar and at least a portion of the insulating intervention sleeve have flexibility to accommodate a non-linear intervention channel. A hardness of the insulating connecting sleeve is greater than that of the insulating puncture sleeve. The biopsy needle bar is accommodated in the outer sleeve with a distal end fixedly connected to the depth adjustment portion through an inner portion of the handle body. The ultrasonic probe is accommodated in the biopsy needle bar, having a distal end fixedly connected to the depth adjustment portion through the inner portion of the handle body. A proximal end of the handle body is fixedly connected to the insulating connecting sleeve. The depth adjustment portion is movably connected to the handle body to drive the biopsy needle bar or the ultrasonic probe to move relative to the outer sleeve.

The biopsy and ultrasound combined device of the invention has the following beneficial effects: the ultrasonic probe is accommodated in the biopsy needle bar, and the biopsy needle bar is accommodated in the outer sleeve. The depth adjustment portion is arranged to be movably connected to the handle body, and fixedly connected to the ultrasonic probe and the biopsy needle bar, respectively, so that the ultrasonic probe, the outer sleeve and the biopsy needle bar are all free to move, thereby facilitating accurate positioning for a puncture direction by the ultrasonic probe before a puncture, and further facilitating confirming that the puncture penetrates a target tissue after the puncture. In addition, since the biopsy needle bar and at least a portion of the insulating intervention sleeve have flexibility and a hardness of the insulating connecting sleeve is greater than that of the insulating puncture sleeve, the smoothness of the puncture process is thereby facilitated and the success rate of biopsy sampling is improved while the accuracy of the biopsy result is increased.

Preferably, the biopsy needle bar has a flexible needle bar and a sampling needle that are connected to each other, and the sampling needle is a rigid structure with a smooth outer side wall. The beneficial effects comprise: the flexible needle bar facilitates adjustment for the direction during a subsequent puncturing process, and the sampling needle having a rigid structure smoothly penetrates the target tissue along a puncturing direction in which the ultrasonic probe is positioned.

Further preferably, the flexible needle bar is a spring tube structure, and an outer side wall surface of the spring tube structure and an outer side wall surface of the sampling needle have a continuous coating to enhance the sealing performance and strength of the biopsy needle bar.

Further preferably, the flexible ultrasound sleeve comprises a first sleeve and a second sleeve, the second sleeve is accommodated in the first sleeve, and one end of the first sleeve and one end of the second sleeve are fixedly connected to the depth adjustment portion. The first sleeve is a flexible plastic sleeve, and the second sleeve is a metal spring tube.

Further preferably, the ultrasonic probe further comprises an operation segment, an embedding member, and an energy transfer medium. The operation segment is fixedly connected to the other end of the flexible ultrasound sleeve, and the embedding member is fixedly connected to the flexible ultrasound sleeve to form a sealing space between the embedding member and the flexible ultrasound sleeve. The operation segment is accommodated in the sealing space, and the energy transfer medium is accommodated in the sealing space while immersing the operation segment.

Preferably, the depth adjustment portion comprises a needle bar adjustment member. The needle bar adjustment member is movably connected to the handle body and has a proximal end fixedly connected to the distal end of the biopsy needle bar to drive the biopsy needle bar to move relative to the outer sleeve or the ultrasonic probe.

Further preferably, an outer side wall of the needle bar adjustment member is disposed with a first scale structure to assist in adjusting a moving displacement of the biopsy needle bar relative to the outer sleeve or the ultrasonic probe.

Further preferably, the handle body has a first cavity structure. The proximal end of the needle bar adjustment member penetrates the distal end of the handle body to enter into the first cavity structure and move in an extension direction of the first cavity structure, thereby driving the biopsy needle bar to move.

Further preferably, the depth adjustment portion further comprises an ultrasound adjustment member. The ultrasound adjustment member is movably connected to the needle bar adjustment member, and the distal end of the ultrasonic probe penetrates the handle body and the needle bar adjustment member to be fixedly connected to the ultrasound adjustment member.

Further preferably, the handle body also has a limit member. One end of the limit member is fixedly connected to an outer side wall of the handle body, and the other end of the limit member has an engagement structure to be removably fixedly connected to the needle bar adjustment member or the ultrasound adjustment member.

Further preferably, an outer side wall of the ultrasound adjustment member is disposed with a second scale structure to assist in adjusting a moving displacement of the ultrasonic probe relative to the outer sleeve or the biopsy needle bar.

Further preferably, the needle bar adjustment member has a second cavity structure therein to accommodate a proximal end of the ultrasound adjustment member. The ultrasound adjustment member moves in an extension direction of the second cavity structure, thereby driving the ultrasonic probe to move.

### BRIEF DESCRIPTION OF THE DRAWINGS

FIG. 1 is a structural view of a biopsy and ultrasound combined device of the invention;

FIG. 2 is a sectional view of a proximal end of the outer sleeve portion shown in FIG. 1;

FIG. 3 is a schematic structural diagram of another biopsy and ultrasound combined device according to some embodiments of this invention;

FIG. 4a is a schematic structural diagram of a limit member shown in FIG. 3;

FIG. 4b is a front view of the distal end of the first handle body shown in FIG. 3;

FIG. 4c is a schematic diagram of an assembly structure of an elastic ring at the distal end of the first handle body shown in FIG. 4a;

FIG. 5 is a structural view of an upper surface of a first moving rod shown in FIG. 3;

FIG. 6a is a view showing a first operation state between the insulating intervention sleeve shown in FIG. 1 and the movable needle bar and the ultrasonic probe shown in FIG. 2;

FIG. 6b is a view showing a second operation state between the insulating intervention sleeve shown in FIG. 1 and the movable needle bar and the ultrasonic probe shown in FIG. 2;

FIG. 6c is a view showing a third operation state between the insulating intervention sleeve shown in FIG. 1 and the movable needle bar and the ultrasonic probe shown in FIG. 2;

FIG. 6d is a view showing a fourth operation state between the insulating intervention sleeve shown in FIG. 1 and the movable needle bar and the ultrasonic probe shown in FIG. 2;

FIG. 6e is a view showing the operation state of sampling of a biopsy needle bar shown in FIG. 1;

FIG. 7 is a structural view of a biopsy needle bar shown in FIG. 2;

FIG. 8 is a structural view of an ultrasonic probe shown in FIG. 2.

### DESCRIPTION OF THE EMBODIMENTS

In order to make objectives, technical solutions, and advantages of the invention clearer, the technical solutions in the invention are described clearly and completely in the following with reference to accompanying drawings in the embodiments of the invention. Apparently, the described embodiments are only a part rather than all of the embodiments of the invention. Based on the embodiments of the invention, all the other embodiments obtained by those with ordinary skill in the art without inventive effort are within the scope of the invention. Unless otherwise mentioned, all technical and scientific terms used herein have the same meaning as commonly understood by one with ordinary skill in the art to which the invention belong. “Comprise” and the like are intended to denote the element or object before the word comprise the listed element or object and thereof equivalents after the word, instead of excluding other elements or objects.

For the problems in the prior art, an embodiment of the invention provides a biopsy and ultrasound combined device, comprising a handle body, a depth adjustment portion, an outer sleeve, a biopsy needle bar, and an ultrasonic probe. An end near a movable end of the outer sleeve is defined as a proximal end.

In some embodiments of the invention, the depth adjustment portion has a needle bar adjustment member and an ultrasound adjustment member.

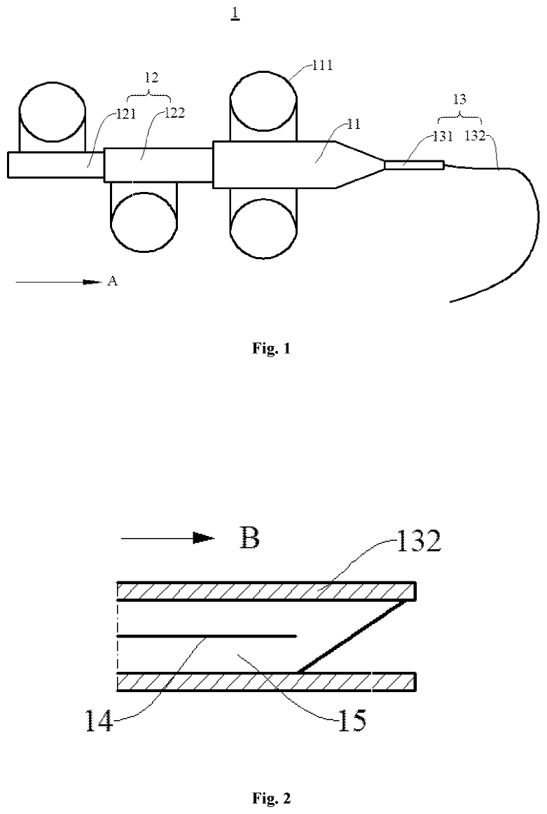

FIG. 1 is a structural view of a biopsy and ultrasound combined device according to some embodiments of the invention. FIG. 2 is a sectional view of a proximal end of the outer sleeve portion shown in FIG. 1.

With reference to FIGS. 1 and 2, the biopsy and ultrasound combined device 1 has a handle body 11, a depth adjustment portion 12, an outer sleeve 13, an ultrasonic probe 14, and a biopsy needle bar 15. The proximal end of the handle body 11 is fixedly connected to a distal end of the outer sleeve 13. The biopsy needle bar 15 is accommodated in the outer sleeve 13, and a distal end of the biopsy needle bar 15 is fixedly connected to the depth adjustment portion 12 through an inside of the handle body 11. The ultrasonic probe 14 is accommodated in the biopsy needle bar 15, and a distal end of the ultrasonic probe 14 is fixedly connected to the depth adjustment portion 12 through an inside of the handle body 11 The proximal end of the ultrasonic probe 14 is provided with an ultrasound transducer (not shown). The depth adjustment portion 12 is movably connected to the handle body 11 to drive the biopsy needle bar 15 or the ultrasonic probe 14 to move relative to the outer sleeve 13.

With reference to FIG. 1, the depth adjustment portion 12 comprise a needle bar adjustment member 122 and an ultrasound adjustment member 121 that are movably connected to each other. The ultrasound adjustment member 121 is movably connected to the needle bar adjustment member 122 to drive the ultrasonic probe 14 to move relative to the outer sleeve. The needle bar adjustment member 122 is movably connected to the handle body 11 to drive the biopsy needle bar 15 to move relative to the outer sleeve 13.

Referring to FIG. 1, a grip portion 111 is fixedly connected to a side wall of the handle body 11. The grip portion 111 comprises a hollow ring structure (not shown in the figure). The other side of the side wall of the needle bar adjustment member 122 and the ultrasound adjustment member 121 is also fixedly connected with other grip portions having the same structure as the grip part 111.

In some embodiments of the invention, an operator inserts and holds a hollow ring structure of the handle body 11 (not shown in the figure) with fingers of one hand, and inserts and holds the needle bar adjustment member 122 or any one of the hollow ring structures of ultrasound adjustment member 121 (not shown in the figure) with fingers of the other hand, and thus is capable of moving the needle bar adjustment member 122 or the ultrasound adjustment member 121 in the A direction or the opposite direction of the A direction.

The outer sleeve 13 has an insulating intervention sleeve 131 and an insulating connecting sleeve 132 that are fixedly connected to each other. A distal end of the insulating connecting sleeve 131 is fixedly connected to a proximal end of the handle body 11. A hardness of the insulating connecting sleeve 131 is greater than that of the insulating intervention sleeve 132 to strengthen the connection relationship between the handle body 11 and the insulating intervention sleeve 132, so that the insulating intervention sleeve 132 may be smoothly and stably fed into the intervention channel in subsequent use operations.

In some embodiments of the invention, the ultrasound biopsy combined device performs a biopsy sampling on a target tumor in a human body through the intervention channel, which is a natural passage or an auxiliary intervention channel of the human body.

In some specific embodiments of the invention, the natural passage of the human body is an esophagus or a trachea, and the auxiliary intervention channel is an auxiliary channel that reaches the vicinity of the target tumor through the subcutaneous puncturing.

In some embodiments of the invention, the insulating intervention sleeve 132 is a flexible tube having insulating properties, which on the one hand ensures the using safety of the operator, and on the other hand is beneficial to move along the nonlinear intervention channel to reach the vicinity of the target tissue. Specifically, the insulating intervention sleeve 132 has a radius of curvature of 5-15 mm.

The length of the insulating intervention sleeve 132 may be adaptively adjusted according to the length of the intervention channel.

In some specific embodiments of the invention, the insulating intervention sleeve 132 is a flexible polyetheretherketone tube.

In some embodiments of the invention, the ultrasonic probe 14 is flexible with a movable end provided with an ultrasound transducer. Specifically, the ultrasonic probe 14 has a radius of curvature of 5-15 mm.

In some embodiments of the invention, the needle bar adjustment member is movably connected to the handle body and has a proximal end fixedly connected to the distal end of the biopsy needle bar to drive the biopsy needle bar to move relative to the outer sleeve.

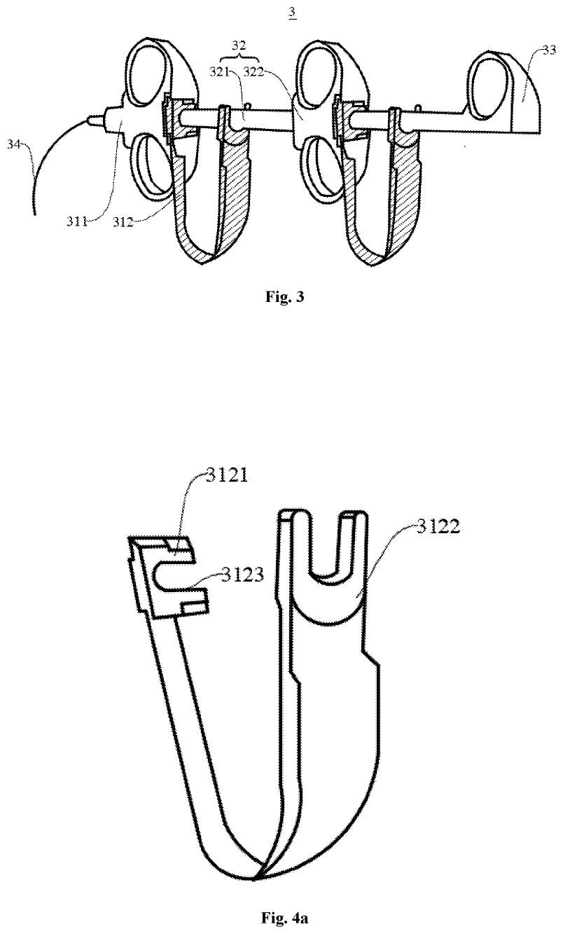

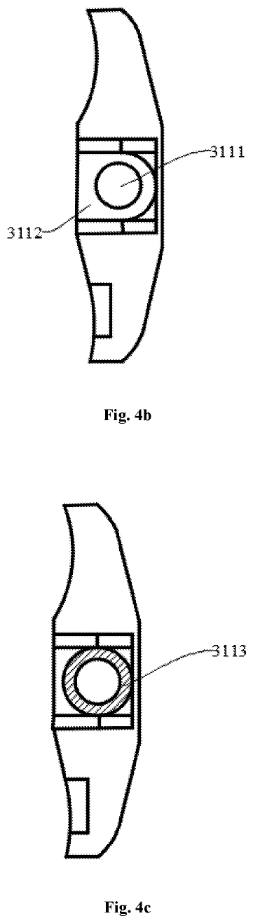

FIG. 3 is a schematic structural diagram of another biopsy ultrasound combined device according to some embodiments of the present invention. FIG. 4a is a schematic structural diagram of a limit member shown in FIG. 3. FIG. 4b is a front view of the distal end of the first handle body shown in FIG. 3. FIG. 4c is a schematic diagram of the assembly structure of the elastic ring at the distal end of the first handle body shown in FIG. 3.

Referring to FIGS. 1, 3 and 4a, one of the main differences between a first biopsy ultrasound combined device 3 and the biopsy ultrasound combination device 1 shown in FIG. 1 lies in that the limit members 312 having the same structure are set both between a first handle body 311 and a first needle bar adjustment member 32 of the first biopsy ultrasound combined device 3 and between the first needle bar adjustment member 32 and a first ultrasound adjustment member 33.

Taking the limit member 312 between the first handle body 311 and the first needle bar adjustment member 32 as an example, a bottom cover 3121 at one end of the limit member 312 is fixedly connected to the distal end of the first handle body 311. That is, an end far from the free end of a first insulating intervention sleeve 34, and the other end has a latching structure 3122 to be detachably and fixedly connected to the first moving rod 321 of the first needle bar adjustment member 32.

In some embodiments of the invention, after the operation of the first needle bar adjustment member 32 is completed, the latching structure 3122 is first latched on the first moving rod 321 so that the first needle bar adjustment member 122 does not undergo relative movement with respect to the first handle body 11, and then the first ultrasound adjustment member 33 is operated, so that the ultrasonic probe 14 is operated at the same time, so that the biopsy needle bar 15 is not moved, and the operation safety of the first biopsy ultrasound combined device 3 is improved.

In some example of the invention, the bottom cover 3121 facing a side of the distal end of the first handle body 311 is detachably and fixedly corresponding to the distal end of the first handle body 311.

Referring to FIGS. 3 and 4a-4c, a U-shaped groove 3112 is provided adjacent to the distal end of the first handle body 311, and the U-shaped groove 3112 is provided with a hole 3111 to communicate with a first cavity (not shown in the figure) penetrating through the first handle body 311 in the first handle body 311. The first cavity (not shown in the figure) is used to accommodate at least a portion of the first moving rod 321.

The bottom cover 3121 comprises a U-shaped hollow area 3123, so that the bottom cover 3121 is fixedly connected to the distal end of the first handle body 311 which, at the same time, makes an arc-shaped portion of the U-shaped hollow area 3123 engage with the first moving rod 321.

Moreover, the first handle body 311 further comprises an elastic ring 3113. The elastic ring 3113 is placed in the U-shaped groove 3112 and the inner diameter of the elastic ring 3113 is adapted to the outer diameter of the first moving rod 321, so that the elastic ring 3113 is sleeved on the first moving rod 321. The inner diameter of the elastic ring 3113 does not exceed the diameter of the hole 3111, so that the first needle bar adjustment member 32 can move relative to the first handle body 311 in the extending direction of the first moving rod 321.

In some embodiments of the invention, the first ultrasound adjustment member 33 is movable connected to the needle bar adjustment member 32. The needle bar adjustment member 32 has a second cavity structure therein to accommodate a proximal end of the ultrasound adjustment member 33. The ultrasound adjustment member 33 moves in an extension direction of the second cavity structure, thereby driving the ultrasonic probe to move relative to the outer sleeve.

With reference to FIGS. 1 and 3, the structure at the distal end of a holding operation portion 322 of the needle bar adjustment member 32 is the same as that of the first handle body 311, and the structure of the first ultrasound adjustment member 33 differs from that of the first needle bar adjustment member 32 in that the first needle bar adjustment member 32 has a second cavity structure (not shown) penetrating the inside, while the first ultrasound adjustment member 33 is a solid structure. For the connection relationship between the first ultrasound adjustment member 33 and the first needle bar adjustment member 32, refer to the above-mentioned connection relationship between the first needle bar adjustment member 32 and the first handle body 311, and it will not be described herein.

In some embodiments of the invention, an outer side wall of the needle bar adjustment member is disposed with a first scale structure to assist in adjusting a moving displacement of the biopsy needle bar relative to the outer sleeve.

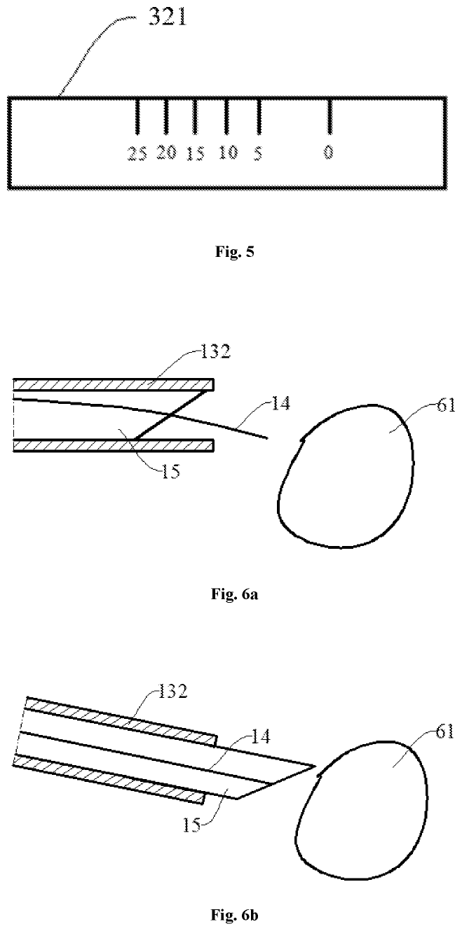

FIG. 5 is a structural view of an upper surface of the first moving rod shown in FIG. 3.

With reference to FIGS. 1 and 5, the upper surface of the first moving rod 321 is provided with a first scale structure (not shown), the first scale structure (not shown) has a scale and a corresponding scale value, and the scale values are sequentially increased in a direction opposite to the A direction shown in FIG. 1.

In some embodiments of the invention, an outer side wall of the ultrasound adjustment member is disposed with a second scale structure to assist in adjusting a moving displacement of the ultrasonic probe relative to the outer sleeve.

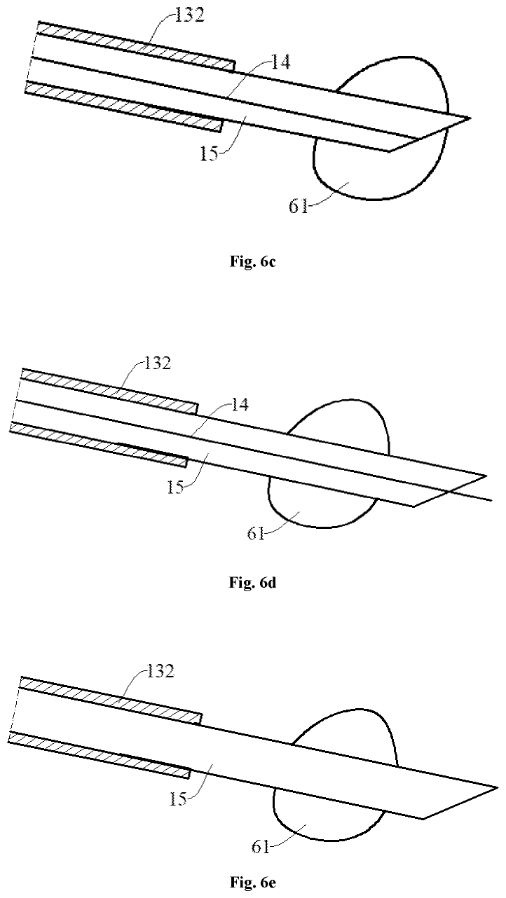

FIGS. 6a-6e are views showing a first operation state between the insulating intervention sleeve shown in FIG. 1 and the biopsy needle bar and the ultrasonic probe shown in FIG. 2. FIG. 6b is a view showing a second operation state between the insulating intervention sleeve shown in FIG. 1 and the movable needle bar and the ultrasonic probe shown in FIG. 2. FIG. 6c is a view showing a third operation state between the insulating intervention sleeve shown in FIG. 1 and the movable needle bar and the ultrasonic probe shown in FIG. 2. FIG. 6d is a view showing a fourth operation state between the insulating intervention sleeve shown in FIG. 1 and the movable needle bar and the ultrasonic probe shown in FIG. 2. FIG. 6e is a view showing the operation state of sampling of a biopsy needle bar shown in FIG. 2.

The following is an example of biopsy sampling of a target tumor of a human body through the natural passage of the human body by the biopsy and ultrasound combined device, and the use process of the biopsy and ultrasound combined device is elaborated in detail:

S0: with reference to FIGS. 1 and 2, an initial state of the biopsy and ultrasound combined device 1 is that the ultrasonic probe 14 and the biopsy needle bar 15 are both accommodated inside the insulating intervention sleeve 132.

S1: with reference to FIGS. 1 and 6a, an operator holds the handle body 11 to feed the insulating intervention sleeve 132 into the human body along the natural passage of the human body to move toward the target tumor 61; at the same time, the operator pushes the ultrasound adjustment member 121 in the A direction such that a movable end of the ultrasonic probe 14 protrudes from a movable end of the insulating intervention sleeve 132, and a puncturing end of the biopsy needle bar 15 is still accommodated within the insulating intervention sleeve 132. Since both the insulating intervention sleeve 132 and the biopsy needle bar 15 have flexibility, the ultrasonic probe 14 may detect the surrounding area and feedback a detection signal through the transducer of the movable end, so as to guide the operator to adjust the direction of intervention of the insulating intervention sleeve 132 at any time.

S2: with reference to FIGS. 1 and 6a, since the ultrasonic probe 14 has flexibility, when the linear distance between the movable end of the ultrasonic probe 14 and the target tumor 61 is 5-6 cm, the operator stops the intervention against the insulating intervention sleeve 132, but adjusts the direction of the movable end of the ultrasonic probe 14 by means of an adjustment device (not shown) externally attached to the handle body 11 to assist in positioning the subsequent puncturing direction of the biopsy needle bar 15.

S3: with reference to FIGS. 1 and 6b, the operator completes the positioning of the puncturing direction through the ultrasonic probe 14 and maintains the ultrasonic probe 14 to stand still, then pushes the needle bar adjustment member 122 in the A direction to move the biopsy needle bar 15 toward the target tumor 61 in the puncturing direction until the movable end of the biopsy needle bar 15 is flush with the movable end of the ultrasonic probe 14.

Meanwhile, since the insulating intervention sleeve 132 has flexibility, when the puncturing direction is not in the same line as a central axis direction of the insulating intervention sleeve 132, the insulating intervention sleeve 132 may drive the biopsy needle bar 15 together for adaptive flexible adjustment while the biopsy needle bar 15 is moving until the central axis direction of the insulating intervention sleeve 132 coincides with the puncturing direction, so as to facilitate the subsequent puncturing process of the biopsy needle bar 15 to proceed smoothly.

S3: with reference to FIGS. 1 and 6c, the operator simultaneously pushes the needle bar adjustment member 122 and the ultrasound adjustment member 121 in the A direction, so that the movable end of the biopsy needle bar 15 is always flush with the movable end of the ultrasonic probe 14 during the puncturing for the target tumor 61 along the puncturing direction by the biopsy needle bar 15, and the ultrasonic probe 14 detects the surrounding area and feedbacks the detection signal at any time through the transducer of the movable end to guide the biopsy needle bar 15 to completely penetrate the target tumor 61, thereby ensuring that two ends of the tissue sample taken by the biopsy needle bar 15 are normal tissues and the middle of the tissue sample is the tumor tissue to be subjected to pathological analysis, so as to guarantee the accuracy of the biopsy results.

S4: with reference to FIGS. 1 and 6d, when the biopsy needle bar 15 penetrates the target tumor 61, the operator stops the adjustment of the needle bar adjustment member 122, so that the biopsy needle bar 15 may not move relative to the target tumor 61. Then, the ultrasound adjustment member 121 is further pushed in the A direction, so that the movable end of the ultrasonic probe 14 protrudes from the movable end of the biopsy needle bar 15, and the surrounding area is detected and the detection signal is fed back at any time through the transducer at the movable end of the ultrasonic probe 14 to further ensure that the biopsy needle 15 does indeed penetrate the target tumor 61.

S5: with reference to FIGS. 1, 6d and 6e, the operator pulls the ultrasound adjustment member 121 to an extreme position in the direction opposite to A, so that the movable end of the ultrasonic probe 14 is away from the movable end of the biopsy needle bar 15. Then, the ultrasound adjustment member 121 is reciprocatingly moved in the A direction and the direction opposite to A, thereby driving the biopsy needle bar 15 to perform reciprocated sampling on the target tumor 61 in the puncturing direction. After the reciprocated sampling, the operator holds the handle body 11 to withdraw the outer sleeve 13 from the natural body passage of the human body, and collects the tissue sample in the biopsy needle bar 15.

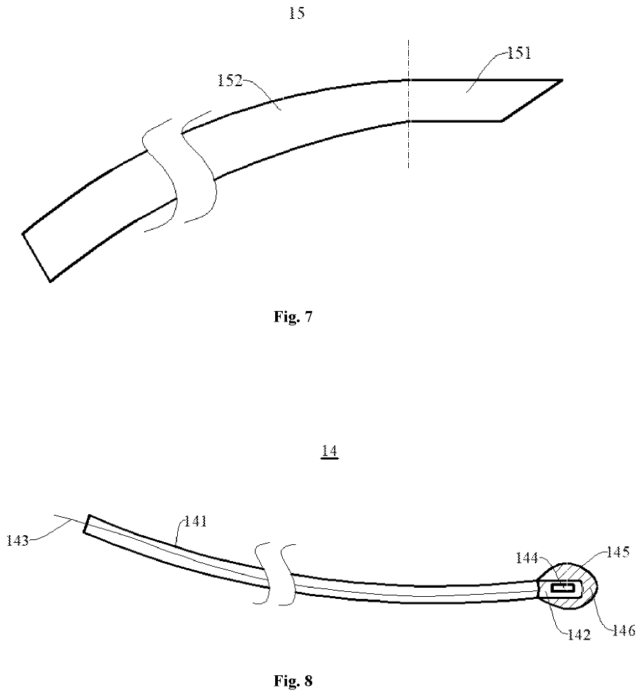

FIG. 7 is a structural view of a biopsy needle bar shown in FIG. 2. With reference to FIG. 7, the biopsy needle bar 15 has a flexible needle bar 152 and a sampling needle 151 that are connected to each other. The sampling needle 151 is a rigid structure with a smooth outer wall, which is advantageous for puncture and sampling.

In some embodiments of the invention, the flexible needle bar 152 is a spring tube structure, and an outer surface of the biopsy needle bar 15 has a continuous coating to enhance the sealing performance and strength of the biopsy needle bar 15.

In other embodiments of the invention, the flexible needle bar 152 is a flexible tube, which is a metal spring tube or a flexible medical plastic tube. In some specific embodiments of the invention, the metal elastic tube is a titanium alloy spring tube.

FIG. 8 is a structural view of an ultrasonic probe shown in FIG. 2.

With reference to FIG. 8, the ultrasonic probe 14 has a flexible ultrasound sleeve 141, an operation segment 142, an inner core 143, and an ultrasound transducer 144. The inner core 143 is accommodated inside the flexible ultrasound sleeve 141. One end of the operation segment 142 is fixed to the flexible ultrasound sleeve 141 by welding, and a signal line (not shown) and a shield line (not shown) of the inner core 143 are fixedly connected to two electrodes (not shown) of the ultrasound transducer 144 by soldering or bonding. An outer surface of the operation segment 142 is opened with a mounting recess (not shown) for disposing the ultrasound transducer 144.

With reference to FIGS. 1 and 8, a distal end of the ultrasound adjustment member 121 is for fixedly connecting an energy generating device (not shown), and the energy generating device (not shown) is fixedly connected to a distal end of the ultrasonic probe 14 to deliver energy to the movable end of the ultrasonic probe 14.

In some embodiments of the invention, the distal end of the ultrasound adjustment member 121 is detachably and fixedly connected to the energy generating device (not shown).

In some embodiments of the invention, with reference to FIG. 8, the ultrasonic probe 14 further comprises an embedding member 145, and the embedding member 145 is a sealing hose with one end open. The open end of the embedding member 145 is fixedly connected to the flexible ultrasound sleeve 141, and accommodates the operation segment 142 in a sealing space. The energy transfer medium 146 fills the sealing space to submerge the operation segment 142 to facilitate the operation segment 142 to obtain a clear image. The energy transfer medium 146 is water, ultrasoundally conductive oil or ultrasoundally conductive jelly.

In some embodiments of the invention, the flexible ultrasound sleeve 141 is composed of a first sleeve and a second sleeve. The second sleeve is accommodated in the first sleeve, the first sleeve is a plastic sleeve, and the second sleeve is a metal spring tube. Both the plastic sleeve and the metal spring tube are flexible to further enhance the supporting function of the biopsy needle bar 15, so that the biopsy needle bar 15 may perform stable puncture in the puncturing direction.

In some specific embodiments of the invention, the second sleeve is a medical titanium alloy spring tube, and the first sleeve is a flexible polyetheretherketone sleeve.

While the embodiments of the invention have been described in detail, it will be apparent to those skilled in the art that various modifications and changes can be made to the embodiments. However, it is to be understood that such modifications and variations are within the scope and spirit of the invention as described in the appended claims. Furthermore, the invention described herein is susceptible to other embodiments and may be embodied or carried out in various ways.

What is claimed is:

1. A biopsy sampling method of a target tissue of a human body through a natural passage of the human body, the method comprising:
providing a biopsy and ultrasound combined device, wherein the biopsy and ultrasound combined device comprises:
a handle body;
an outer sleeve, wherein the outer sleeve comprises:
an insulating connecting sleeve having a distal end fixedly connected to a proximal end of the handle body; and
an insulating intervention sleeve having a distal end fixedly connected to a proximal end of the insulating connecting sleeve, wherein at least a portion of the insulating intervention sleeve is flexible, and a hardness of the insulating intervention sleeve is smaller than a hardness of the insulating connecting sleeve;
a biopsy needle bar accommodated in the insulating intervention sleeve, wherein the biopsy needle bar is flexible;
an ultrasonic probe accommodated in the biopsy needle bar; and
a depth adjustment portion movably connected to the handle body to drive the biopsy needle bar, the ultrasonic probe or both to move relative to the outer sleeve, wherein the depth adjustment portion are fixedly connected to a distal end of the biopsy needle bar, the ultrasonic probe or both through an inner portion of the handle body;
holding the handle body to feed the insulating intervention sleeve into the human body along the natural passage of the human body to move toward the target tissue;
pushing the ultrasound adjustment member in a direction toward the target tissue, such that a movable end of the ultrasonic probe protrudes from a movable end of the insulating intervention sleeve, and a puncturing end of the biopsy needle bar is still accommodated within the insulating intervention sleeve;
stopping the insulating intervention sleeve;
adjusting the direction of the movable end of the ultrasonic probe to position a subsequent puncturing direction of the biopsy needle bar toward the target tissue;
pushing the needle bar adjustment member to move the biopsy needle bar toward the target tissue until a movable end of the biopsy needle bar is flush with the movable end of the ultrasonic probe;
simultaneously pushing the needle bar adjustment member and the ultrasound adjustment member until the target tissue is completely penetrated by the biopsy needle bar;
further pushing the ultrasound adjustment member, so that the movable end of the ultrasonic probe protrudes from the movable end of the biopsy needle bar to detect surrounding region;
pulling the ultrasound adjustment member back, so that the movable end of the ultrasonic probe is away from the movable end of the biopsy needle bar; and
withdrawing the outer sleeve from the natural passage of the human body to collect the target tissue in the biopsy needle bar.

2. The method of claim 1, further comprising:
reciprocatingly moving the ultrasound adjustment member to drive the biopsy needle bar to perform reciprocated sampling on the target tumor after the step of pulling the ultrasound adjustment member back and before the step of withdrawing the outer sleeve.

3. The method of claim 1, wherein the insulating intervention sleeve is flexible, so that the insulating intervention sleeve may drive the biopsy needle bar together for adaptively flexible adjustment while the biopsy needle bar is moving until a central axis direction of the insulating intervention sleeve coincides with the puncturing direction to facilitate the subsequent puncturing process of the biopsy needle bar.

4. The method of claim 1, wherein the biopsy needle bar comprises:
a flexible needle bar; and
a sampling needle connected to the flexible needle bar, wherein the sampling needle is a rigid structure with a smooth outer side wall.

5. The method of claim 4, wherein penetrating the target tissue by the sampling needle along the puncturing direction in which the ultrasonic probe is positioned in the step of simultaneously pushing the needle bar adjustment member and the ultrasound adjustment member.

6. The method of claim 1, wherein the flexible needle bar is of a spring tube structure, and an outer side wall surface of the spring tube structure and an outer side wall surface of the sampling needle has a continuous coating to enhance the sealing performance and strength of the biopsy needle bar.

7. The method of claim 1, wherein the ultrasonic probe comprises:
a flexible ultrasound sleeve accommodated in the biopsy needle bar, wherein one end of the flexible ultrasound sleeve is fixedly connected to the depth adjustment portion.

8. The method of claim 7, wherein the flexible ultrasound sleeve comprises:
a first sleeve fixedly connected to the depth adjustment portion, wherein the first sleeve is a flexible plastic sleeve; and
a second sleeve accommodated in the first sleeve and fixedly connected to the depth adjustment portion, wherein the second sleeve is a metal spring tube.

9. The method of claim 7, wherein the ultrasonic probe further comprises:
an embedding member fixedly connected to the flexible ultrasound sleeve to form a sealing space between the embedding member and the flexible ultrasound sleeve;
an operation segment fixedly connected to the flexible ultrasound sleeve and accommodated in the sealing space; and
an energy transfer medium accommodated in the sealing space to immerse the operation segment.

10. The method of claim 7, wherein the depth adjustment portion comprises:
a needle bar adjustment member movably connected to the handle body, wherein the needle bar adjustment member is fixedly connected to the biopsy needle bar to drive the biopsy needle bar to move relative to the outer sleeve or the ultrasonic probe.

11. The method of claim 10, wherein an outer side wall of the needle bar adjustment member having a first scale structure disposed thereon to assist in adjusting a moving displacement of the biopsy needle bar relative to the outer sleeve or the ultrasonic probe.

12. The method of claim 11, wherein the handle body has a first cavity structure, and a proximal end of the needle bar adjustment member penetrates a distal end of the handle body to enter into the first cavity structure and move in an extension direction of the first cavity structure, thereby driving the biopsy needle bar to move.

13. The method of claim 11, wherein the depth adjustment portion further comprises:
an ultrasound adjustment member movably connected to the needle bar adjustment member, wherein the ultrasound adjustment member is fixedly connected to the ultrasonic probe penetrating the handle body and the needle bar adjustment member.

14. The method of claim 13, wherein the handle body further comprises a limit member, one end of the limit member is fixedly connected to an outer side wall of the handle body, and the other end of the limit member has an engagement structure to be removably fixedly connected to the needle bar adjustment member or the ultrasound adjustment member.

15. The method of claim 13, wherein an outer side wall of the ultrasound adjustment member has a second scale structure disposed thereon to assist in adjusting a moving displacement of the ultrasonic probe relative to the outer sleeve or the biopsy needle bar.

16. The method of claim 13, wherein the needle bar adjustment member has a second cavity structure therein to accommodate a proximal end of the ultrasound adjustment member, and the ultrasound adjustment member moves in an extension direction of the second cavity structure, thereby driving the ultrasonic probe to move.

17. The method of claim 1, wherein sampling the target tissue includes along an auxiliary intervention channel of the human body in the step of holding the handle body to feed the insulating intervention sleeve into the human body.
